(19) United States
(12) Patent Application Publication
(10) Pub. No.: US 2024/0382236 A1
(43) Pub. Date: Nov. 21, 2024

(54) EXTERNAL FIXATION SYSTEMS AND METHODS OF USE

(71) Applicant: Paragon 28, Inc., Englewood, CO (US)

(72) Inventors: Kenneth Allan ROGGOW, Denver, CO (US); Garrett Jeffrey LIPKER, Arvada, CO (US); Daniel CAPELIN, Denver, CO (US); Dimitri PROTOPSALTIS, Memphis, TN (US)

(73) Assignee: Paragon 28, Inc., Englewood, CO (US)

(21) Appl. No.: 18/787,174

(22) Filed: Jul. 29, 2024

Related U.S. Application Data
(63) Continuation-in-part of application No. PCT/US2023/061367, filed on Jan. 26, 2023.
(60) Provisional application No. 63/304,236, filed on Jan. 28, 2022.

(51) Int. Cl. A61B 17/62; A61B 17/64

(52) U.S. Cl. CPC A61B 17/62; A61B 17/6425; A61B 17/6475

(57) ABSTRACT

An external fixation system includes an upper fixation element including a semicircular geometry, and an intermediate fixation element including a circular geometry, in which the intermediate fixation element is releasably coupled with the upper fixation element. The system also includes a lower fixation element including a substantially U-shaped geometry terminating at a first end point and a second end point, in which the lower fixation element is releasably coupled with the intermediate fixation element, and an anterior fixation element including a semicircular geometry. The anterior fixation element is also fixedly coupled with the first and second end points of the lower fixation element. The system also may include at least one strut member that is releasably coupled with the intermediate fixation element and the lower fixation element. Also disclosed is a joint having a first component, a second component and an intermediate component positioned therebetween to allow for pivotable motion.

### CROSS REFERENCE TO RELATED APPLICATION

This application is a bypass continuation-in-part of PCT Application No. PCT/US2023/061367, filed Jan. 26, 2023, and entitled “External Fixation Systems and Methods of Use,” which claims priority benefit under 35 U.S.C. § 119(e) of U.S. Provisional Application No. 63/304,236 filed Jan. 28, 2022, entitled “External Fixation Systems and Methods of Use” which are both incorporated herein by reference in their entirety.

### TECHNICAL FIELD

The present disclosure relates to fixation systems for orthopedic applications. Further, the present disclosure related to external fixation systems to be implemented in treating various acute and chronic orthopedic conditions. More specifically, but not exclusive, the present disclosure relates to external fixation systems to be implemented in orthopedic conditions of the foot and/or ankle.

### BACKGROUND OF THE INVENTION

Many currently available fixation systems and corresponding methods of use do not completely address the needs of patients. Additionally, many currently available fixation systems and corresponding methods of use fail to account for anatomical properties of patients, for example the anatomy of the foot, ankle, and surrounding anatomical structures thereof and accordingly may not produce the most favorable outcome for said patients.

### SUMMARY OF THE INVENTION

The present disclosure is directed toward surgical instruments, implants, and methods directed to arthroplasty procedures.

A first aspect of the present disclosure includes an external fixation system. The system includes an upper fixation element including a semicircular geometry, and an intermediate fixation element including a circular geometry, wherein the intermediate fixation element is releasably coupled with the upper fixation element. The system also includes a lower fixation element including a substantially U-shaped geometry terminating at a first end point and a second end point, wherein the lower fixation element is releasably coupled with the intermediate fixation element, and an anterior fixation element including a semicircular geometry, wherein the anterior fixation element is fixedly coupled with the first and second end points of the lower fixation element. The system also includes at least one strut member releasably coupled with the intermediate fixation element and the lower fixation element.

According to one aspect of the present disclosure, the external fixation system, the at least one strut member includes a pair of strut members.

According to one aspect of the present disclosure, the at least one strut member includes a first end having a first threaded portion to facilitate threadable coupling, a second end having a second threaded portion to facilitate threadable coupling, and a central portion positioned between the first end and the second end.

According to one aspect of the present disclosure, the at least one strut member includes a gross adjustment mechanism positioned adjacent the second end and the central portion of the strut member. The gross adjustment mechanism includes a collar positioned diametrically about a threaded rod, and a sleeve positioned diametrically outward from the collar and the threaded rod.

According to one aspect of the present disclosure, the collar of the at least one strut member is threadably engaged with a threading of the threaded rod such that manipulation of the collar about the threading translates the collar along the threaded rod.

According to one aspect of the present disclosure, the sleeve of the at least one strut member is translatable along the threaded rod.

According to one aspect of the present disclosure, the at least one strut member also includes a locking nut translatable along the length of the threaded rod and positioned adjacent the sleeve.

According to one aspect of the present disclosure, the at least one strut member also includes a tube, wherein the threaded rod is translatable such that at least a portion of the threaded rod is disposed within the tube.

According to one aspect of the present disclosure, the tube of the at least one strut member includes a plurality of markings on an external surface thereof indicating a length of the at least one strut member based on the position of at least a portion of the threaded rod disposed at least partially within the tube.

According to one aspect of the present disclosure, the at least one strut member also includes a fine adjustment mechanism actuatable by rotating at least a portion of the fine adjustment mechanism, wherein rotational manipulation corresponds to predetermined length adjustments to the strut member.

A second aspect of the present disclosure includes a joint of an external fixation system. The joint includes a first component with a first longitudinal axis, and a second component with a second longitudinal axis. The first and second components are manipulatable from a first position in which the first and second longitudinal axes are concentric, to a second position in which the first and second longitudinal axes intersect to form an oblique angle with one another.

According to one aspect of the present disclosure, the first component includes an elongate member having first threading on an external surface thereof extending from a terminal end to a central portion thereof, and a body disposed opposite the elongate member from the terminal end.

According to one aspect of the present disclosure, the body includes a substantially spherical geometry with a lateral dimension greater than a diameter of the elongate member.

According to one aspect of the present disclosure, the second component includes a volume extending therethrough, the volume having a first threading disposed on at least a portion of an interior surface thereof.

According to one aspect of the present disclosure, the joint includes an adapter having a second threading disposed on an external surface thereof and configured to engage the first threading of the volume of the second component.

According to one aspect of the present disclosure, the adapter includes a cannulation and third threading disposed on an interior surface of the cannulation, wherein the third threading is configured to threadably engage a complimentary thread of an element of an external fixation system.

According to one aspect of the present disclosure, the volume of the second component is configured to receive at least a portion of the body therein.

According to one aspect of the present disclosure, the second component includes a shoulder adjacent the space configured to retain at least a portion of the body within the space.

According to one aspect of the present disclosure, the first component is releasably couplable with a ring member of an external fixation system, and the second component is releasably couplable with a strut member of an external fixation system.

A third aspect of the present disclosure includes a joint of an external fixation system. The joint includes a first component with a first pair of protrusions defining a first space therebetween, wherein each of the first pair of protrusions includes a first bore extending therethrough, wherein the first bores are coaxial. The joint also includes a second component with a second pair of protrusions defining a second space therebetween, wherein each of the second pair of protrusions includes a second bore extending therethrough, wherein the second bores are coaxial. The joint also includes an intermediate component positioned at least partially within the first and second spaces and having a third bore aligned with the first bores and receiving a first fastener therethrough so as to couple the intermediate component with the first component, and a fourth bore aligned with the second bores and receiving a second fastener therethrough so as to couple the intermediate component with the second component, wherein the first and second components are respectively pivotable about the first and second fasteners.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a perspective view of an exemplary external fixation system, in accordance with the present disclosure;

FIG. 2 is a front view of the exemplary external fixation system of FIG. 1, in accordance with the present disclosure;

FIG. 3 is top view of the exemplary external fixation system of FIG. 1, in accordance with the present disclosure;

FIG. 4 is a rear view of the external fixation system of FIG. 1, in accordance with the present disclosure;

FIG. 5 is a side view of the external fixation system of FIG. 1, in accordance with the present disclosure;

FIG. 6 is a bottom view of the exemplary external fixation system of FIG. 1, in accordance with the present disclosure;

FIG. 7 is a first side view of an exemplary strut member for implementation with the external fixation system of FIG. 1, in accordance with the present disclosure;

FIG. 8 is a second side view of the exemplary strut member of FIG. 7 for implementation with the external fixation system of FIG. 1, in accordance with the present disclosure;

FIG. 9 is a first perspective view of the exemplary strut member of FIG. 7 for implementation with the external fixation system of FIG. 1, in accordance with the present disclosure;

FIG. 10 is a second perspective view of the exemplary strut member of FIG. 7 for implementation with the external fixation system of FIG. 1, in accordance with the present disclosure;

FIG. 11 is a side cross-sectional view of the exemplary strut member of FIG. 7 for implementation with the external fixation system of FIG. 1, in accordance with the present disclosure;

FIG. 12 is a perspective view of the exemplary strut member of FIG. 7 coupled with two joints, in accordance with the present disclosure;

FIG. 13 is an alternate perspective view of the exemplary strut member of FIG. 12 coupled with two joints, in accordance with the present disclosure;

FIG. 14 is a perspective view of a joint for implementation with the exemplary strut member of FIG. 7 and the external fixation system of FIG. 1, in accordance with the present disclosure;

FIG. 15 is an alternate perspective view of the joint of FIG. 14 for implementation with the exemplary strut member of FIG. 7 and the external fixation system of FIG. 1, in accordance with the present disclosure;

FIG. 16 is a side cross-sectional view of the joint of FIG. 14 for implementation with the exemplary strut member of FIG. 7 and the external fixation system of FIG. 1, in accordance with the present disclosure;

FIG. 17 is a side cross-sectional view of the exemplary strut member of FIG. 7 coupled with the two joints of FIG. 12 for implementation with the external fixation system of FIG. 1, in accordance with the present disclosure;

FIG. 18 is a perspective view of an exemplary joint couplable with the exemplary strut member of FIG. 7 for implementation with the external fixation system of FIG. 1, in accordance with the present disclosure;

FIG. 19 is a side view of an exemplary joint couplable with the exemplary strut member of FIG. 7 for implementation with the external fixation system of FIG. 1, in accordance with the present disclosure;

FIG. 20 is a perspective view of an exemplary joint couplable with the exemplary strut member of FIG. 7 for implementation with the external fixation system of FIG. 1, in accordance with the present disclosure;

FIG. 21 is a side view of an exemplary joint couplable with the exemplary strut member of FIG. 7 for implementation with the external fixation system of FIG. 1, in accordance with the present disclosure;

FIG. 22 is a perspective view of an exemplary joint couplable with the exemplary strut member of FIG. 7 for implementation with the external fixation system of FIG. 1, in accordance with the present disclosure;

FIG. 23 is a side view of an exemplary joint couplable with the exemplary strut member of FIG. 7 for implementation with the external fixation system of FIG. 1, in accordance with the present disclosure;

FIG. 24 is a perspective view of an exemplary footplate coupling system for implementation with the external fixation system of FIG. 1, in accordance with the present disclosure;

FIG. 25 is an alternate perspective view of the exemplary footplate coupling system of FIG. 24 for implementation with the external fixation system of FIG. 1, in accordance with the present disclosure;

FIG. 26 is a front view of an exemplary strut member for implementation with the external fixation system of FIG. 1, in accordance with the present disclosure;

FIG. 27 is a rear view of the exemplary strut member of FIG. 26 for implementation with the external fixation system of FIG. 1, in accordance with the present disclosure;

FIG. 28 is a side cross-sectional view of the exemplary strut member of FIG. 27 for use with the external fixation system of FIG. 1, in accordance with the present disclosure;

FIG. 29 is a perspective view of the exemplary strut member of FIG. 26 in use with the external fixation system of FIG. 1, in accordance with the present disclosure; and

FIG. 30 is a side perspective view of the exemplary strut member of FIG. 26 for use with the external fixation system of FIG. 1, in accordance with the present disclosure.

### DETAILED DESCRIPTION OF THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current systems, implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the system, implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the system, implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the system, implants, devices, instrumentation, and methods, and the aspects, components, features and the like thereof, described herein with respect to the right foot may be mirrored so that they likewise function with the left foot. Further, the system, implants, devices, instrumentation, and methods, and the aspects, components, features and the like thereof, disclosed herein are described with respect to the foot for brevity purposes, but it should be understood that the system, implants, devices, instrumentation, and methods may be used with other bones of the body having similar structures.

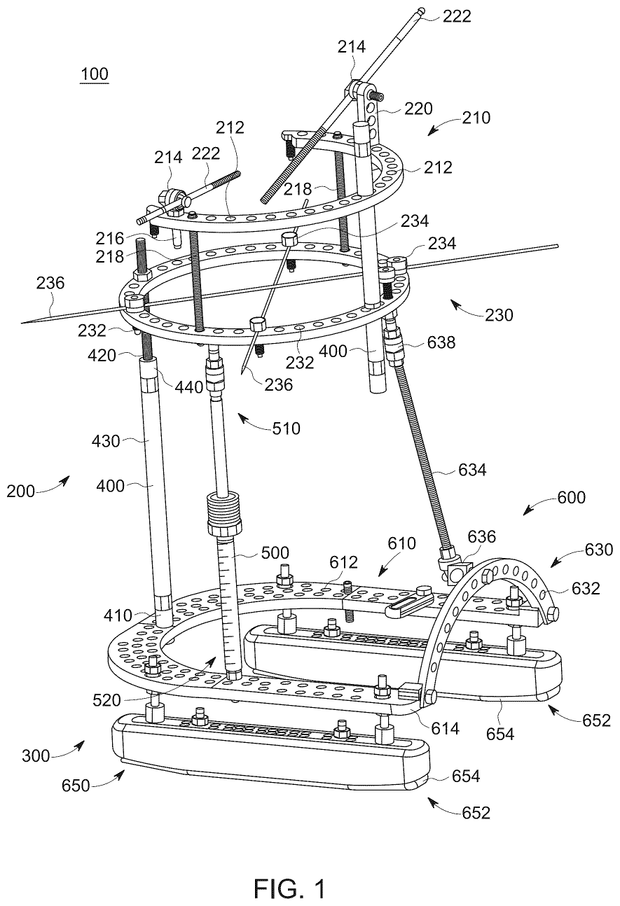

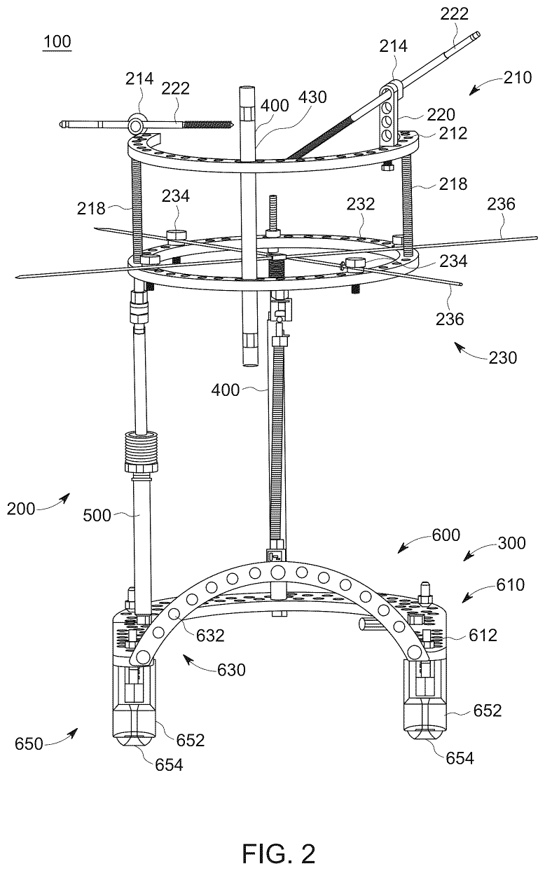

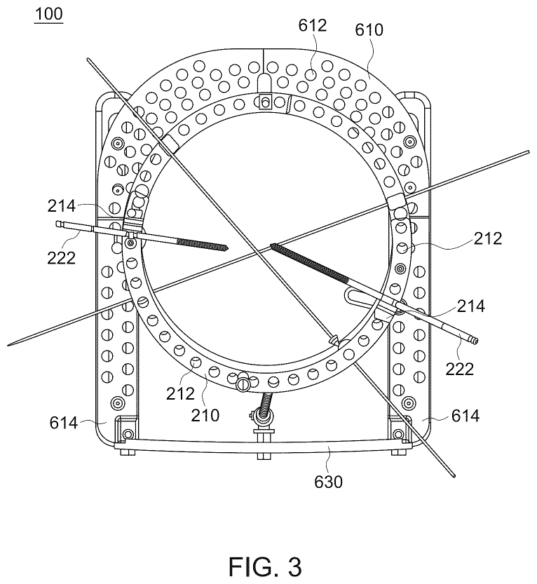

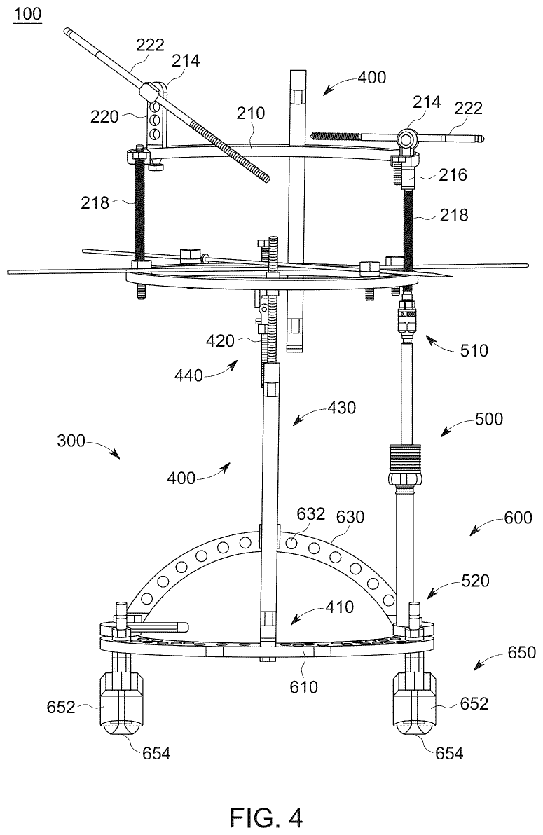

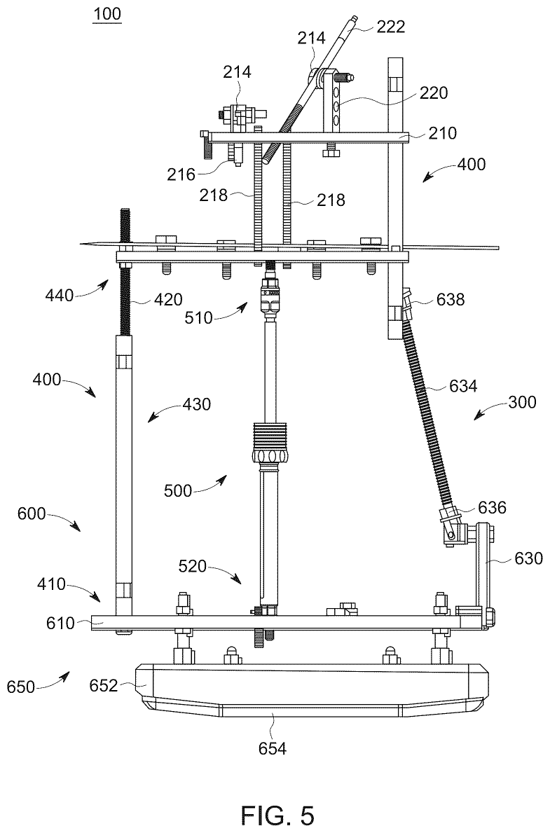

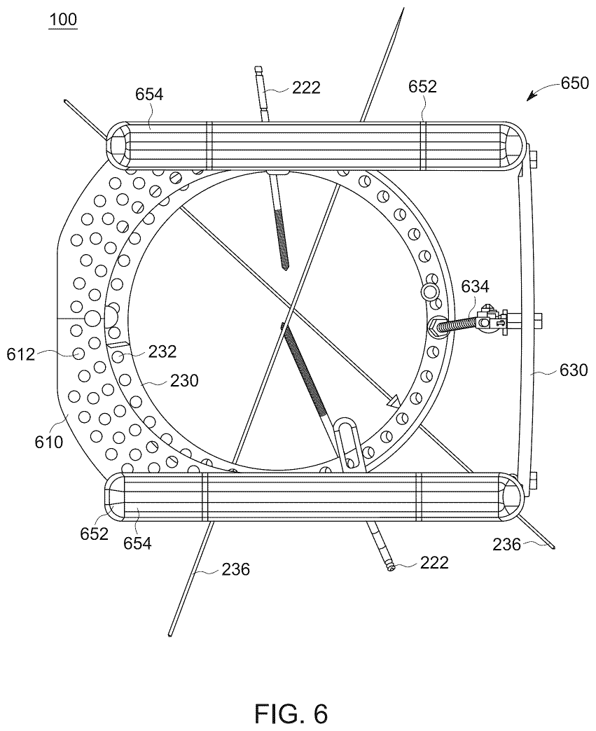

Referring to the drawings, wherein like reference numerals are used to indicate like or analogous components throughout the several views, and with particular reference to FIGS. 1-6, there is illustrated an exemplary embodiment of an external fixation system 100 (referred to hereinafter as “system 100”). The system 100 may include various components including but not limited to those shown and described herein. The system 100 and components thereof may also be provided to a physician in an unassembled state such that the physician may select desired components to implement in order to provide external fixation for a patient using the system 100. Further, the system 100 may include one or more of the components shown and described herein, where a physician may desire to use multiple of one or more components or elect not to use one or more components when selecting components for assembling the system 100 so as to best provide external fixation for a patient.

The system 100 is shown to include an upper portion 200 and a lower portion 300. The upper portion 200 and the lower portion 300 may be coupled with each other (e.g., releasably, directly, indirectly, fixedly, etc.) via various components including but not limited to those shown and described herein. The upper portion 200 is shown to include a first ring member 210 and a second ring member 230. In some aspects, the system 100 may include one or more of both the first ring member 210 and the second ring member 230, as well as any other ring members shown and described herein. As shown, the first ring member 210 has a semi-circular geometry (e.g., less than an entire circle). Further, as shown the first ring member 210 includes a substantially hemi-circular geometry (e.g., approximately half of a circle). In some aspects, the first ring member 210 may include a full circular geometry, a less than hemi-circular geometry, or may include other alternate geometries (e.g., u-shaped, etc.). The second ring member 230 is shown to be coupled with the first ring member 210 (e.g., releasably, fixedly, threadably, pivotably, etc.) at least one coupling point. In some aspects, the first ring member 210 may be coupled with the second ring member 230 such that each of the ring members are arranged about a common longitudinal axis. The first and second ring members 210, 230 may also be coupled with one another so as to permit movement (e.g., translating, pivoting, etc.) about one or more axes or coupling points.

The first ring member 210 is shown to include a plurality of holes 212 arranged about the first ring member 210. As shown, the holes 212 are arranged about the circumference or partial circumference of the first ring member 210 and spaced equidistantly from one another and also equidistantly from both the inner and outer edges of the first ring member 210. In some aspects, the holes 212 may be spaced alternatively (e.g., various distances from one another, biased toward the outer or inner edge of the first ring member 210, etc.). The holes 212 are also shown to be substantially circular and of an equal size, however in some aspects one or more of the holes 212 may be of an alternate size (e.g., greater or lesser diameter) and/or may be of an alternate geometry (e.g., hexagon, octagon, etc.). In some aspects, the interior surface of the holes 212 may include a threading configured to receive a complimentary component therein/therethrough. As shown, the first ring member 210 is releasably coupled with the second ring member 230 via one or more structural members 218 configured to be at least partially received within the holes 212 and a plurality of holes 232 disposed about the second ring member 230 (where the holes 232 may be the same as and/or similar to the holes 212). The structural member 218 is shown to have a cylindrical geometry including a threading disposed on an outer surface along the length thereof, where said threading may be received at least partially within the holes 212, 232. In some aspects, the rod 218 may be releasably coupled with the first and second ring members 210, 230 via one or more other coupling components, for example various threaded nuts having a threading complimentary to that of the rod 218.

The first ring member 210 is further shown to include one or more coupling devices 214 releasably coupled with one or more of the holes 212. As shown, the coupling devices 214 include a linear member 216 (e.g., a rigid linear component) disposed on a lower portion thereof configured to be received by the holes 212 and thereby facilitate releasable coupling with the first ring member 210. The coupling devices 214 include at least one cavity (e.g., depression, void, bore, through hole, opening, etc.) configured to receive and facilitate releasable coupling with a rod 222. The upper portion 200 and/or the system 100 may include one or more rods 222, where the one or more rods 222 may be configured to releasably couple with an anatomical structure of a patient (or other hardware component including but not limited to those shown and described here). In some aspects, the rod 222 may include a substantially cylindrical geometry and may further include a threading disposed on at least a portion of the length. The cavity of the coupling devices 214 may be configured to facilitate translation of the rod 222 while maintaining coupling therewith. In some aspects, the first ring member 210 may include an extension 220 configured to releasably couple with both a hole 212 of the first ring member 210 and the coupling device 214 so as to space the coupling device 214 from the first ring member 210 (e.g., elevate the coupling device 214 vertically from a surface of the first ring member 210 such that the rod 222, when coupled with the coupling device 214, may be releasably coupled with at least one anatomical structure of a patient at an angle different than that which may be achieved without the extension 220).

The second ring member 230 is shown to include one or more coupling devices 234 releasably coupled with one or more of the holes 232. As shown, the coupling devices 234 may an extension (e.g., a rigid linear component which may be the same as and/or similar in structure and/or function as the linear member 216) disposed on a lower portion thereof configured to be received by the holes 232 and thereby facilitate releasable coupling with the second ring member 230. The coupling devices 234 include at least one cavity (e.g., depression, void, bore, through hole, opening, etc. which may be the same as and/or similar to that of the coupling devices 214) configured to receive and facilitate releasable coupling with at least a portion of a pin 236 (of which the system 100 may include one or more). The upper portion 200 and/or the system 100 may include one or more pins 236, where the one or more pins 236 may be configured to releasably couple with an anatomical structure of a patient (or other hardware component including but not limited to those shown and described here). The pins 236 may have a similar length and/or geometry to the rods 222 as shown with reference to the first ring member 210, however the pins 236 may have a lesser lateral dimension, lack threading, or include/lack various other structure components. The cavity of the coupling devices 234 may be configured to facilitate translation of the pin 236 while maintaining coupling therewith. In some aspects, the second ring member 230 may include an extension similar to the extension 220 shown with reference to the first ring member 210, where such an extension may be configured to releasably couple with both a hole 232 of the second ring member 230 and the coupling device 234 so as to space the coupling device 234 from the second ring member 230 (e.g., elevate the coupling device 234 vertically from a surface of the second ring member 230 such that the pin 236, when coupled with the coupling device 234, may be releasably coupled with at least one anatomical structure of a patient at an angle different than that which may be achieved without the extension).

The system 100 is shown to include at least one rod member 400. As shown in FIGS. 1-6, the system 100 includes one rod member 400 releasably coupling the first ring member 210 with the second ring member 230. As shown, a housing 430 of the rod member 400 is received by and extends through both a hole 212 and a hole 232 such that the rod member 400 is configured in a plane perpendicular to that of a top or bottom surface of the first and second ring members 210, 230. The system 100 also shows a rod member 400 releasably coupling the second ring member 230 of the upper portion 200 with a third ring member 610 of a ring system 600. The third ring 610 member is shown to include a substantially u-shaped geometry, with said geometry defined by a pair of terminal ends 214 positioned at the end of two substantially parallel linear segments of the u-shape. The third ring member 610 includes a plurality of holes 612 disposed about the third ring member 610, where the size and geometry of the holes 612 may be the same as and/or similar to that of the holes 212, 232. In some aspects, the third ring member 610 may include one or more rows or other configurations/patterns of the holes 612 disposed variously about the third ring member 612.

The rod 400 releasably coupling the second ring member 230 with the third ring member 610 is shown to include a first end 410 of the housing 430 received by one of the holes 612 and releasably coupled therewith/therethrough. The housing 430 is also shown to include a second end 440 opposite the housing 430 from the first end 410, with the second end 440 including a threaded rod 420 extending therefrom. The threaded rod 420 is shown to be received by one of the holes 232 and may be coupled with the second ring member 230 via the hole 232 using one or more other coupling components (for example, a threaded nut). The housing 430 may include a bore extending therethrough from the first end 410 to the second end 430, where at least a portion of said bore may include a threading complimentary to that of the threaded rod 420. In some aspects, the housing 430 may releasably couple with one or more threaded rods 420 via the first and second ends 410, 440 (and/or the threaded bore therewithin and therebetween) so as to facilitate alternative means of releasably coupling with various ring members or other components of the system 100 as well as provide various height configurations (depending on how much the rod member 420 is disposed within/extending from the housing 430).

The ring system 600 is shown to include a fourth ring member 630 having a semi-circular geometry (e.g., less than an entire circle). Further, as shown the fourth ring member 630 includes a substantially hemi-circular geometry (e.g., approximately half of a circle), although in some aspects the ring member 630 may have a geometry that includes less than half of a circle. In some aspects, the fourth ring member 630 may include other alternate geometries (e.g., u-shaped, etc.). The fourth ring member 630 includes a plurality of holes 632 disposed about the fourth ring member 630, where the size and geometry of the holes 632 may be the same as and/or similar to that of the holes 212, 232 and 612. The fourth ring member 630 is shown to be releasably coupled with the terminal ends 614 of the third ring member 610 such that the surfaces adjacent the holes 632 fall in planes substantially perpendicular to the surfaces adjacent the holes 612. In some aspects, the fourth ring member 630 may be configured so such planes are positioned at substantially oblique angles. As shown, coupling members (e.g., bolts or other coupling components) are each received within one of the holes 632 (which, in the exemplary embodiment of FIGS. 1-6, are the holes nearest the terminal ends of the fourth ring member 630) and extend at least partially through the holes 632 such that the coupling components are received at least partially within threaded bores disposed within the terminal ends 614 of the third ring member 610. As shown in FIGS. 1-6, the fourth ring member 630 is coupled with the third ring member 610 such that the fourth ring member 630 is not pivotable or otherwise movable relative to the third ring member 610 without decoupling the pair of coupling components from the terminal ends 614.

The fourth ring member 630 is also shown to be releasably coupled with the second ring member 230 via a rod 634, a first coupling device 636, and a second coupling device 638. The first coupling device 636 is shown to be releasably coupled with one of the holes 632. Such releasably coupling may include one or more common coupling components (e.g., bolt, nut, etc.) and may further include at least a portion of the first coupling device 636 and/or said common coupling component being received at least partially within/therethrough the hole 632. As shown, the first coupling device 636 is coupled with a hole 632 that is approximately equidistant the terminal ends of the fourth ring member 630 (e.g., the ends of the fourth ring member 630 are coupled with the terminal ends 614 of the third ring member 610). The first coupling device 636 may include a bearing or one or more other mechanisms configured to facilitate movement (e.g., rotation, pivoting, etc.) of at least a first portion of the first coupling device 636 relative to a second portion of the first coupling device 636, where the second portion of the first coupling device 636 is directly coupled with the fourth ring member 630. The first coupling device 636 is shown to be releasably coupled with the rod 634 (e.g., where the rod 634 may have at least a portion thereof received by at least a portion of the first coupling device 636), with the rod 634 including a threading disposed at least partially along a length thereof. In some aspects, the rod 634 may be the same as and/or similar to the structural member 218. The rod 634 is shown to be releasably coupled with the second coupling device 638, which is in turn coupled with the second ring member 230. In some aspects, the first coupling device 636 and the second coupling device 638 may be the same or may be similar to one another. Further, similar to the first coupling device 636, the second coupling device 638 may include a bearing or one or more other mechanisms configured to facilitate movement (e.g., rotation, pivoting, etc.) of at least a first portion of the first second device 638 relative to a second portion of the second coupling device 638, where the second portion of the second coupling device 638 is directly coupled with the second ring member 230.

The lower portion 300 is further shown to include a ground contact system 650 (e.g., “the system 650”), as shown in FIGS. 1-6. The system 650 includes a pair of support members 652, where each of said support members is configured to have an elongated, substantially rectangular geometry. The support members 652 are shown to releasably coupled with the third ring member 610 which, as shown in the exemplary embodiment of FIGS. 1-6, includes the support members 652 releasably coupled with the legs of the u-shaped geometry of the third ring member 610 such that the support members 652 are disposed beneath the third ring member 610. As shown, each of the support members are releasably coupled with the third ring member 610 at two different coupling points (e.g., with one coupling point adjacent the terminal ends 614 and the other coupling point at an opposite point of the leg of the u-shape). In some aspects, the support members 652 may include one or more coupling elements extending vertically therefrom and as shown in FIGS. 1-6, configured to be received at least partially within and/or through the holes 612 of the third ring member 610. In some aspects, the support members may include one or more holes thereon/therein which may be the same as and or similar to the holes 212, 232, 612, 632 and are configured to facilitate coupling between the support members 652 and other members of the system 100 (e.g., when implemented in conjunction with other common coupling components). In some aspects, such coupling elements extending from the support members 652 may be coupled with an upper portion of the support members 652 and may also be common coupling elements the same as and/or similar to other elements shown and described herein (e.g., threaded rods, nuts, bolts, etc.). Similarly, the coupling elements of the support members 652 may be releasably couplable with the holes 612 of the third ring member 610 when implemented in conjunction with other common coupling elements including but not limited to those mentioned previously herein. In some aspects, the support members 652 may be translatable relative to the third ring member 610 (for example, in the anterior-posterior direction) without decoupling from the third ring member 610. Although not shown, this translation or other adjustment (e.g., height added to the system 100 by adding the support members 652) by loosening and/or tightening one or more common coupling components releasably coupled with the support members 652.

The support members 652 are each shown to include a ground contact member 654 coupled (releasably or otherwise) with a bottom (e.g., inferior, distal, etc.) surface of each of the support members 652. In some aspects, each ground contact member 654 is shown to be coupled with each of the support members 652 via common coupling components implemented in conjunction with the one or more holes disposed on or in the support members 652 (where said holes may be the same as and/or similar to the holes 212, 232, 612, 632 shown and described previously herein). The ground contact members 654 may also be coupled with the support members 652 via an adhesive substance applied to one or both of the support members 652 and/or the ground contact members 654. Although not shown, the ground contact members 654 may include a textured surface on a bottom surface thereof (e.g., a tread) configured to prevent slipping of a patient in weight-bearing scenarios. For example, such a texture may include various ribbing or other textures configured to increase friction with a contact surface (e.g., the ground when walking) and/or disperse moisture or other potentially slippery substances from underneath the ground contact member 654 during the gait cycle. Further, such a texture may also include text reading “Paragon 28”, “P28”, “Monkey Sticks”, “Monkey Rings” or other textual components. Similarly, the texture may also include various logos, for example those used commercially by Paragon 28, Inc. The ground contact members 654 may also be configured to be replaceable. For example, if a patient were to wear down a texture and/or ground contact member, the patient or a physician may decouple a used pair of ground contact members 654 and replace with a new set of ground contact members 654. In some aspects, the system 100 may include various ground contact members 654 having various widths, heights, textured surfaces or other characteristics.

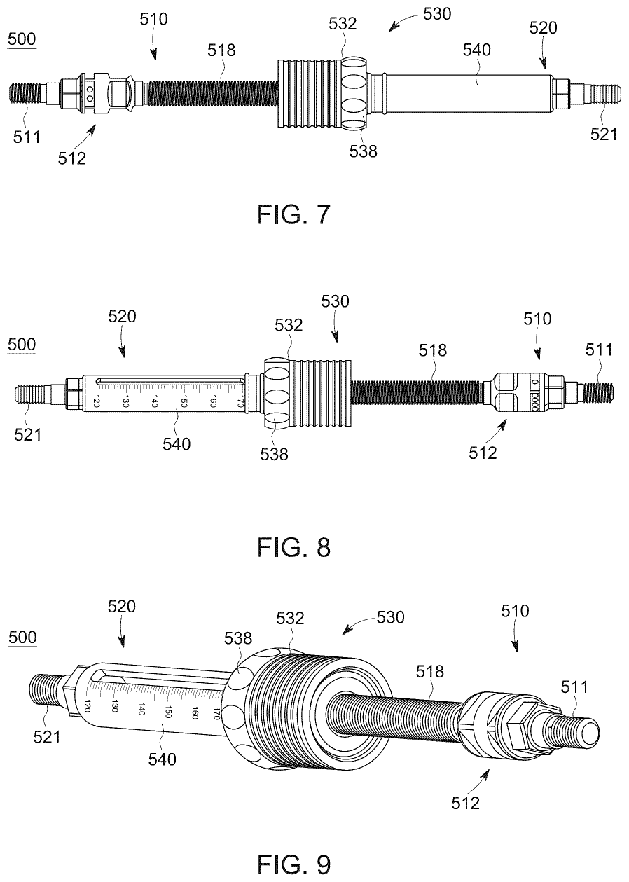

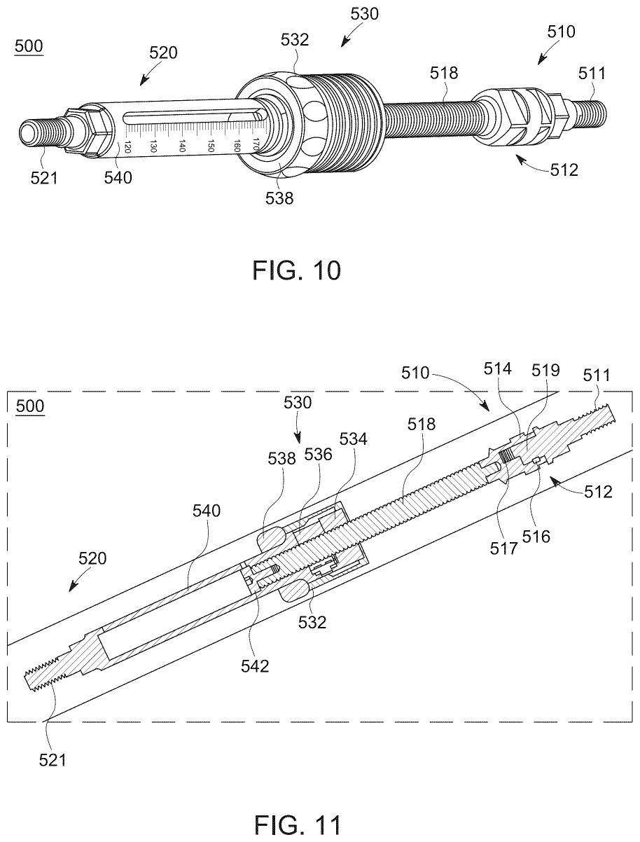

The system 100 is also shown to include a strut 500, shown in FIGS. 1-6 in conjunction with other components of the system 100 and in FIGS. 7-11 independent of the system 100. In some aspects, the system 100 may include more than one strut 500 and, further, the system 100 may be built by a physician using more than one strut 500. As shown in FIGS. 1-6, the strut 500 is releasably coupled with the second ring member 230 and the third ring member 610. However, in alternate embodiments (or build configurations at the discretion of a physician), the strut 500 may be coupled with one or more alternate components of the system 100. The strut 500 is shown to be releasably couplable with one of the holes 232 of the second ring member 230 as well as one of the holes 612 of the third ring member 610, although the strut 500 may also couple with other similar hole (or other coupling devices including but not limited to those shown and described herein). Once a user has coupled the strut 500 with, for example, the second ring member 230 and the third ring member 610, the strut 500 may be manipulated (e.g., actuated, adjusted, etc.) so as to change the distance between the second ring member 230 and the third ring member 610.

The strut 500 is shown to include a first end 510, a second end 520, and a central portion 530, where the first end 510 is opposite the strut 500 from the second end 520 and the central portion 530 is disposed between the first and second ends 510, 520. The strut 500 is releasably couplable with one or more other components of the system 100 (or in some aspects, other external fixation systems/components), for example the second ring member 230 and the third ring member 610 as shown and described previously herein. The first and second ends 510, 520 include a respective first and second threaded portion 511, 521. The threaded portions 511, 521 are both shown to have substantially cylindrical geometries and may include one or more other characteristics similar to other linear structural components of the system 100 shown and described herein (e.g., structural member 218).

The second end 520 of the strut 500 includes a gross adjustment mechanism disposed at or near the central portion 530 which, when the strut 500 is implement by a physician, will likely be manipulated prior to the fine adjustment mechanism shown and describe subsequently herein. The central portion 530 of the strut 500 is shown to include a sleeve 532 positioned diametrically outward from a collar 534, where the collar 534 is arranged about the outer surface of the rod 518. In some aspects, an inner surface of the collar 534 (which abuts the outer surface/threading of the rod 518) may include a complimentary threading, teeth, or other textures or engagement means to facilitate engagement between the collar 534 and the rod 518. The collar 534 may be translatable about the length of the rod 518 or at least a portion thereof. The sleeve 532 may also be moveable (e.g., translatable) about the collar 534 and/or the rod 518 such that, when manipulated, all of, a portion of, or none of the collar 534 may be disposed diametrically between the rod 518 and the collar 534. In some aspects, the sleeve 532 may be open at one or more ends and include a substantially cylindrical geometry. Further, in some aspects the sleeve 532 may include a threading or other texture on an exterior surface thereof.

The central portion 530 is further shown to include a nut 536 configured to be at least partially adjacent to (e.g., positioned next to, contacting, engaging, disposed within a geometric portion of, etc.) the collar 534. As shown in FIG. 11, the nut 536 is arranged diametrically about the rod 518 and within the sleeve 532. Further, the nut 536 is engaged with (e.g., in contact with) the rod 518 as well as the collar 534. The central portion 530 of the strut 500 is also shown to include a locking nut 538 and a tube 540. The locking nut 538 may include grooves or texture on an exterior surface thereof to facilitate gripping and/or manipulation thereof. In some aspects, the tube 540 may be integral with the second threaded portion. The tube 540 is positioned such that it may receive at least a portion of the rod 518 into an inner portion thereof (where the tube 540 includes a substantially cylindrical geometry about at least a portion thereof and further includes a bore extending at least partially therein). As shown, the tube 540 includes various length markings on an outer surface thereof that are positioned adjacent to an elongated window. This elongated window provides fluid communication between the bore of the tube 540 and an outer surface thereof. The locking nut 538 is arranged adjacent the sleeve 532 (e.g., diametrically about at least a portion of the rod 518) and includes a threading on an inner surface thereof configured to engage with a complimentary threading disposed on at least a portion of an exterior surface of the tube 540. The tube 540 is shown to include a limiting element 542, at least a portion of which includes a substantially circular/cylindrical geometry which is the same as and/or similar to the rod 518 and/or the bore of the tube 540 (e.g., complimentary to, configured to contact, to be received by or disposed in).

The strut 500 may be adjusted in a gross manner (e.g., larger increments relative to the fine adjustment mechanism/method shown and described subsequently herein) by a user or physician to make relatively large adjustments to the length of the strut 500. In order to adjust the length of the strut 500, and with reference to FIG. 11, a user may manipulate the locking nut 538 from right to left by rotating (e.g., screwing) the locking nut 536 about the threading disposed on the outer portion of the tube 540 with which the locking nut is engaged. Such movement of the locking nut 536 breaks contact between the sleeve 532 and the locking nut 536, thus permitting the sleeve 532 to be slid in the same direction as the locking nut 536 when manipulated (e.g., right to left as shown). This movement of the sleeve 532 disengages an inner surface (which may include a projection/protrusion configured to contact the nut 536) of the sleeve from an outer surface of the nut 536 which had previously been biased by the sleeve 532 such that the nut 536 was mechanically engaged with at least a portion of the rod 518. The disengagement of the nut 536 from the rod 518 permits movement of the rod 518 relative to the tube 540 (e.g., translation within/along a longitudinal axis thereof). This movement of the rod is limited by the limiting element 542 disposed within the tube 540. When a desired length of the strut 500 is achieved, the aforementioned components may be replaced in the reverse order of that described previously to enable gross adjustment of the strut 500 (e.g., the nut 536 re-engages with the threading of the rod 518, the sleeve 532 is manipulated such that at least a portion of the inner surface of the sleeve 532 contacts at least a portion of the outer surface of the nut 536, and the locking nut 538 is manipulated along the exterior threading of the tube 540 until the locking nut 538 abuts the sleeve 532 thus mechanically retaining the strut 500 at the desired length).

The limiting element 542 is configured to prevent over-adjustment (e.g., over expansion) of the strut 500, for example by manipulating the rod 518 to the right with reference to FIG. 11. The limiting element 542 has a greater lateral dimension than that of the opening of the bore of the tube 540 and, accordingly mechanically limits the lengthening of the strut 500 as translation of the rod 518 to lengthen the strut 500 will ultimately be mechanically limited when the limiting element 542 (which is coupled with an end of the rod 518) cannot be withdrawn from the bore of the tube 540. Adjustment of the strut 500 in a shortening manner can be accomplished by taking the same steps outlined herein, but translating the rod 518 until it contacts a terminal end of the bore of the tube 540 thus mechanically limiting the shortening of the strut 500. While adjusting the strut 500 using the gross adjustment mechanism, at least a portion of the rod 518 may be visible within the elongated opening of the tube 540 such that an end portion of the rod 518 will be positioned adjacent the length-indicating markings on the exterior surface of the tube 540. The length-indicating marking of which the end portion of the rod 518 is adjacent corresponds to a length of the strut 500 at the current adjustment.

The first end 510 of the strut 500 includes an actuator 512 arranged adjacent the first threaded portion 511. The actuator 512 is configured to facilitate fine length adjustment of the strut 500 and is further configured to be rotatable about a rod 518, where the rod 518 includes a threading disposed along at least a portion of the length thereof and is received at least partially within a bore 514 of the actuator (where the bore 514 may include a threading). In some aspects the actuator 512 may be configured such that a specified amount of rotation of the actuator 512 about the rod 518 (e.g., degrees or rotations) corresponds a linear length adjustment (e.g., millimeters). For example, one full rotation of the actuator 512 about the rod 518 may correspond to an increase or decrease in the length of the strut 500 by 1 mm, depending on which direction the actuator 512 is rotated. In some aspects, such calibration may be indicated by one or more markings on an outer surface of the actuator 512. As shown, the actuator 512 includes a double-ended arrow where each end of the arrow indicates a direction of rotation. Further, the actuator 512 is shown to include one or more incrementing indicators (e.g., dots) configured to guide a user in rotation of the actuator 512 to increase or decrease the length of the strut 500 by rotating in one of the directions indicated by the double-ended arrow. The actuator 512 may include one or more flats or other surfaces on an outer portion thereof configured to facilitate gripping and rotation of the actuator 512 about the rod 518.

The actuator 512 is further configured to include a biasing element 516 configured to provide feedback to a physician or other user in response to the rotation of the actuator 512. The feedback provided by the biasing element 516 may include audible feedback (e.g., clicking) as well as haptic feedback (e.g., a physician may feel incremental clicking while holding and actuating the actuator 512). The biasing element 516, as shown in the exemplary embodiment of FIG. 11, includes a spring 517 and a ball member 519. The spring 517 may be compressed when the actuator 512 is rotated about the rod 518 and may further compress and expand in conjunction with said rotation of the actuator 512 to provide one or both of the haptic and/or audible feedback to a user. The ball member 519 may be configured to facilitate rotation of the actuator 512 about the rod 518 in a fashion similar to the function of ball bearings, for example contacting one or more components and facilitating movement of one or both of said components. In some aspects, the aforementioned incremental indicators may also provide visual feedback to a user as the actuator 512 is rotated about the rod 518 indicating either an increase or a decrease in length of the strut 500 depending on direction of rotation. In some aspects, the spring 517 may be replaced with an alternative resilient member and/or the ball member 519 may be replaced with an alternative substantially round component with similar mechanical and kinematic properties. In implementing the system 100 and one or more struts 500 in conjunction with the system 100, a physician may perform gross adjustment of the strut 500 prior to fine adjustment of the strut 500. That is to say that the fine adjustment of the strut 500 via the actuator 512 and components thereof may be performed to determine or achieve a final desired length of the strut 500.

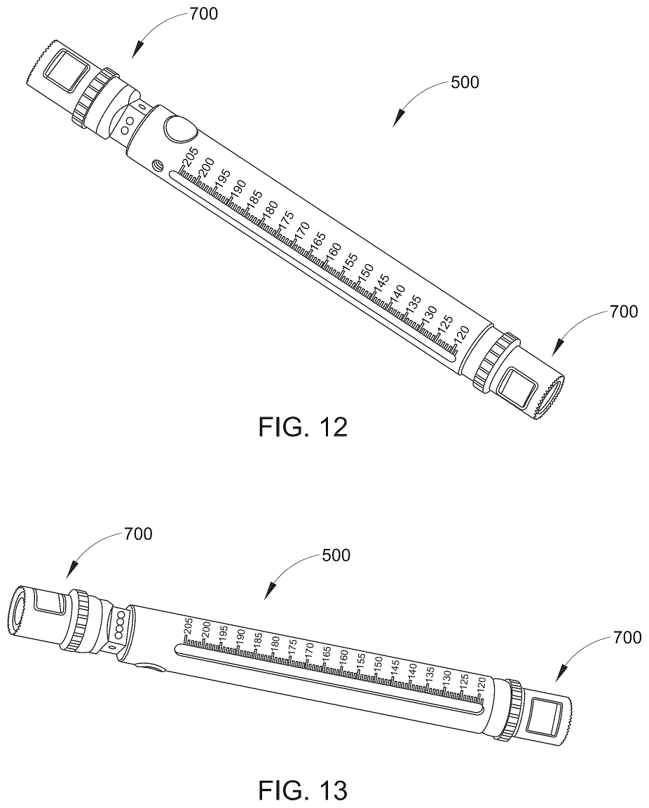

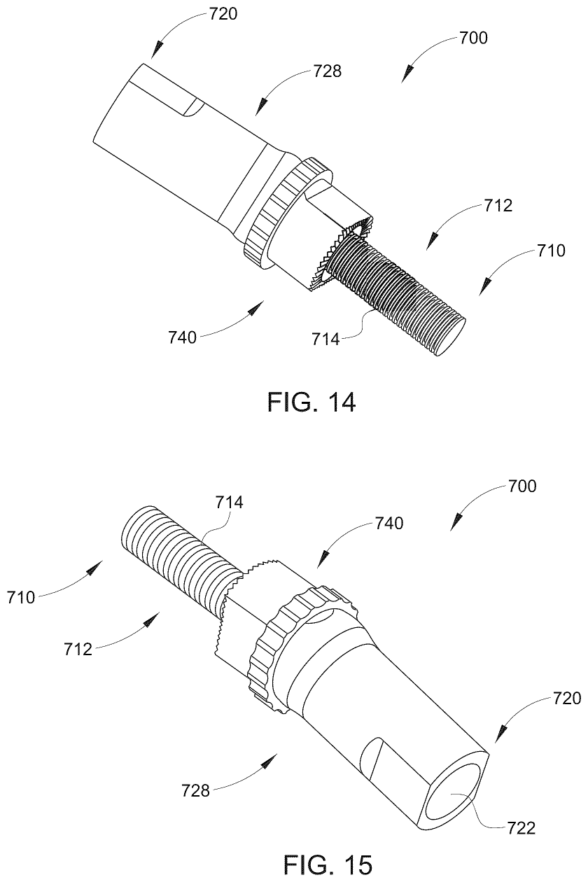

Referring now to FIGS. 12-17, a modular ball joint 700 (referred to hereinafter as “joint 700”) is shown, according to an exemplary embodiment. The joint 700 is couplable with one or both ends of the strut 500 as shown in FIGS. 12-13 and 17, or may be couplable with other struts and/or components of the system 100 as shown in FIG. 1. One or more of the joint 700 may be implemented variously within the system 100, for example coupled with one or both ends of the strut 500, or coupled with one or both ends of other rigid components of the system 100. The joint 700 is shown to be modular in that it has multiple pieces/components that are couplable with another to form the joint 700.

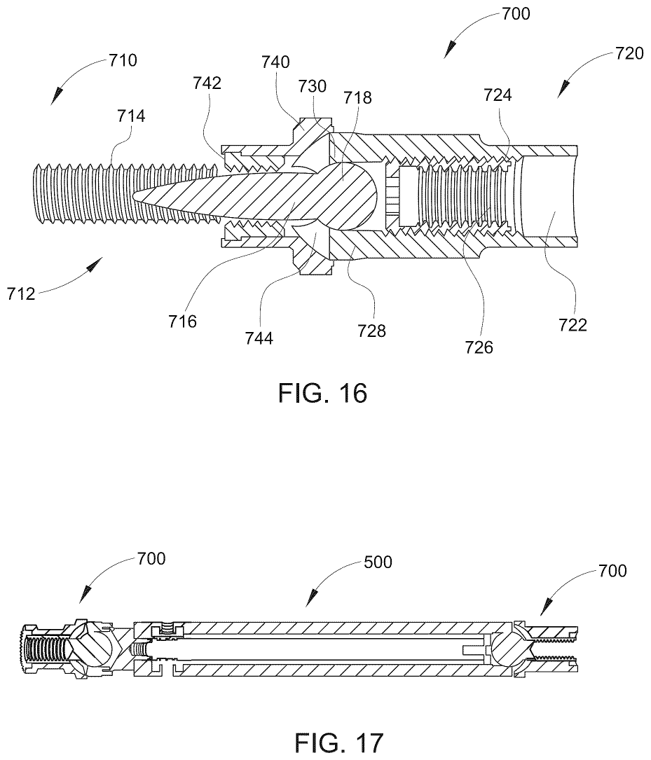

The joint 700 is shown to include a first portion 710 having a first end arranged substantially opposite the joint 700 from a second portion 720 having a second end, where the first and second portions 710, 720 are manipulatable (e.g., actuatable, etc.) relative to one another such that respective longitudinal axes of the first and second portions 710, 720 may be positioned coaxially or such that said axes form an oblique angle with one another. The first portion 710 includes an elongate member 712 which, as shown, has a substantially cylindrical geometry and includes a threading 714 disposed on an exterior surface thereof. The elongate member 712 includes a body 718 configured opposite the elongate member 712 from the first end, which is shown in FIGS. 16-17 as a sphere-shaped geometry (and shown to have a greater lateral dimension than that of the elongate member 712). However, it should be understood that in some aspects, the body 718 may include alternate geometries in other embodiments, for example ellipsoid geometries. The second portion 720 is shown to include a volume or space 722 extending along a longitudinal axis thereof from an opening adjacent the aforementioned second end and along the length of the second portion 720. As shown, the volume 722 has a substantially cylindrical geometry and includes a threading 724 disposed on at least a portion of an internal surface thereof. Further, the second portion 720 is shown to include an adapter 726 threadably coupled with the threading 724 via a threading of the adapter 726 such that the adapter 726 is disposed within the volume 722. The adapter 726 has a cylindrical, cannulated geometry and includes a threading on an interior surface thereof in addition to the threading on the exterior surface which is engaged with the threading 724. The adapter 726 may be configured to facilitate compatibility with various struts and coupling components thereof, for example a strut the same as or similar to the strut 500 so as to receive a threadably coupling portion of the strut 500 within the cannulated portion of the adapter 726 that would be undersized and thus unable to engage with the threading 724 of the second portion 720. In some aspects, the adapter 726 may not be required for coupling between the second portion 720 and a corresponding coupling feature of a strut (or other component of the system 100).

The second portion 720 includes a first housing portion 728 positioned substantially opposite the second portion 720 from the opening of the volume 722 configured to receive a coupling portion of a strut or other component. As shown, the first housing portion 728 is positioned to abut a second housing portion 740 of the first portion 710 and retain at least a portion of the body 718 within the volume 722. The first housing portion 728 includes a shoulder 730 extending circumferentially about an end portion of the volume 722 so as to promote the retention of at least a portion of the body 718 therein. The second housing portion 740 is shown to include a threading 742 disposed on at least a portion of an inner surface thereof configured to engage with the threading 714 of the elongate member 712. The second housing portion 740 also includes a volume or space 744, shown in the exemplary embodiment of FIG. 16 as a concavity. The first housing portion 728 is configured with a lesser lateral dimension than the volume 744 such that manipulation of the first housing portion 728 relative to the second housing portion 740 (e.g., moving the longitudinal axes of the first and second portions 710, 720 to an oblique angle) biases at least a portion of the first housing portion 728 within the volume 744 of the second housing portion 740. When applied to the system 100, a pair of joints 700 may be coupled with the strut 500 at opposite ends thereof to facilitate non-linear (e.g., off-axis) coupling of the strut 500 with one or more of the ring members or other structures. For example, when viewed from above, the strut 500 may be coupled with a lower circular ring member and an upper circular member (e.g., component 230 of FIG. 1) via a pair of joints 700, with the first joint 700 coupling the strut 500 at a 9 o'clock position on the lower circular ring member and the second joint 700 coupling the strut 500 at a 6 o'clock position on the upper circular ring member. Such a configuration is an example of a non-linear, “off-axis” configuration of the strut 500 which may also be classified as the strut 500 being implemented in the system 100 (or other similar systems) in a position such that the strut 500 forms an oblique angle with the horizontal and vertical planes.

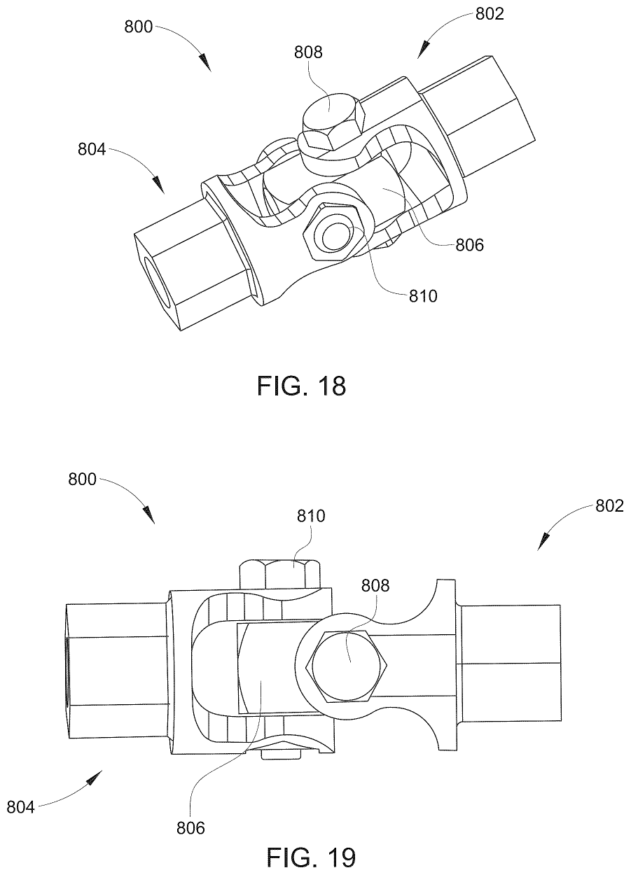

Referring now to FIGS. 18-19, a universal joint 800 (referred to hereinafter as “joint 800”) is shown. Similar to the joint 700, the joint 800 may be releasably (e.g., threadably, etc.) coupled with one or both ends of the strut 500 (or similar) or other rigid linear components of the system 100 or other similar systems. The joint 800 may be configured to facilitate the implementation of the system 100 or similar with the strut 500 or other components in “off-axis” configurations including, for example, the “off-axis” configuration described previously with reference to the strut 500 and the joint 700. The joint 800 is shown to include a first component 802 and a second component 804 configured opposite one another. In some aspects, the first and second components 802, 804 may be substantially similar and/or the same component. Each of the first and second components 802, 804 are shown to include a pair of protrusions extending parallel to one another therefrom with each having a hole extending therethrough and coaxial with the hole on the opposite protrusion. The joint 800 further includes an intermediate component 806 configured to have lateral dimensions lesser than the width between the inner surface of the protrusions on each of the first and second components 802, 804. As shown, the intermediate component 806 includes a substantially rectanguloid (e.g., rectangular prism) geometry, but may also include alternate geometries in some embodiments. In some aspects, the intermediate component 806 may be solid or may be at least partially hollow.

The intermediate component 806 is shown to include a pair of bores extending therethrough at opposite ends of the intermediate component 806 such that longitudinal axes thereof lie in planes orthogonal to one another. The intermediate component 806 is shown to be positioned such that at least a portion of the intermediate component 806 is disposed in the space between the pair of protrusions on both the first and second components 802, 804. Further, a first fastener 808 is configured to couple the intermediate component 806 with the first component 802 and retain the intermediate component 806 in said position at least partially within the space between the protrusions of the first component 802. The first fastener 808 is configured to be received and extend therethrough the hole on the first protrusion of the first component 802, through a bore of the intermediate component 806, and through the hole of the second protrusion of the first component 802, thus retaining the intermediate component 806 in the aforementioned position. Similarly, a second fastener 810 is configured to couple the intermediate component 806 with the second component 804 and retain the intermediate component 806 in said position at least partially within the space between the protrusions of the second component 804. The second fastener 810 is configured to be received and extend therethrough the hole on the first protrusion of the second component 804, through a bore of the intermediate component 806, and through the hole of the second protrusion of the second component 804, thus retaining the intermediate component 806 in the aforementioned position (similar to the first component 802). As shown, the first and second components 802, 804 are approximately positioned 90 degrees offset from one another (e.g., out of phase, etc.) when in the engaged orientation with the intermediate component 806. Accordingly, the first and second components 802, 804 are pivotable about the respective first and second fasteners 808, 810 so as to facilitate movement, manipulation, and various positioning of the joint 800.

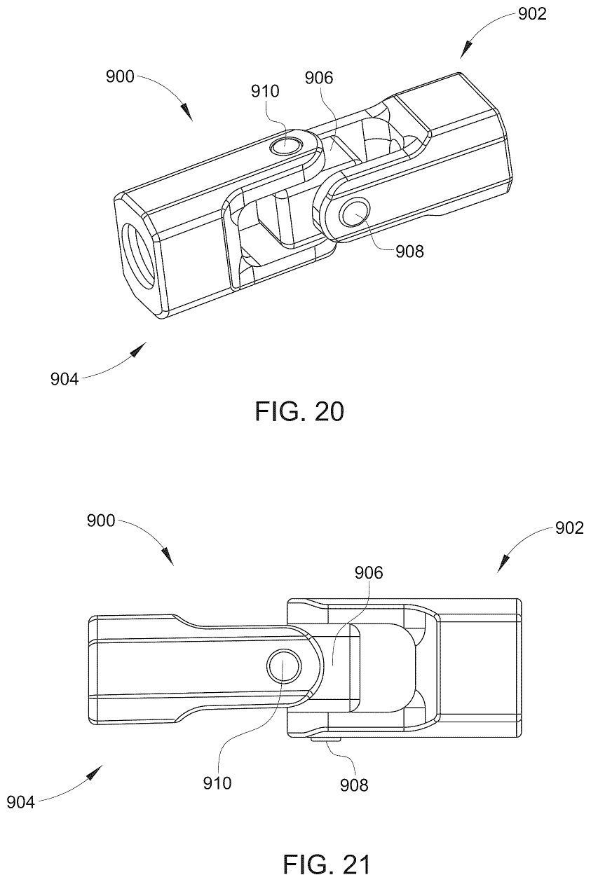

Referring now to FIGS. 20-21, a universal joint 900 (referred to hereinafter as “joint 900”) is shown. Similar to the joints 700, 800, the joint 900 may be releasably (e.g., threadably, etc.) coupled with one or both ends of the strut 500 (or similar) or other rigid linear components of the system 100 or other similar systems. The joint 900 may be configured to facilitate the implementation of the system 100 or similar with the strut 500 or other components in “off-axis” configurations including, for example, the “off-axis” configuration described previously with reference to the strut 500 and the joint 700. The joint 900 is shown to include a first component 902 and a second component 904 configured opposite to one another. In some aspects, the first and second components 902, 904 may be substantially similar and/or the same component. Each of the first and second components 902, 904 are shown to include a pair of protrusions extending parallel to one another therefrom with each having a hole extending therethrough and coaxial with the hole on the opposite protrusion. The joint 900 further includes an intermediate component 906 configured to have lateral dimensions lesser than the width between the inner surface of the protrusions on each of the first and second components 902, 904. As shown, the intermediate component 906 includes a substantially rectanguloid (e.g., rectangular prism) geometry, but may also include alternate geometries in some embodiments. In some aspects, the intermediate component 906 may be solid or may be at least partially hollow.

The intermediate component 906 is shown to include a pair of bores extending therethrough at opposite ends of the intermediate component 906 such that longitudinal axes thereof lie in planes orthogonal to one another. The intermediate component 906 is shown to be positioned such that at least a portion of the intermediate component 906 is disposed in the space between the pair of protrusions on both the first and second components 902, 904. Further, a first fastener 908 is configured to couple the intermediate component 906 with the first component 902 and retain the intermediate component 906 in said position at least partially within the space between the protrusions of the first component 902. The first fastener 908 is configured to be received and extend therethrough the hole on a first protrusion of the first component 902, through a bore of the intermediate component 906, and through the hole of the second protrusion of the first component 902, thus retaining the intermediate component 906 in the aforementioned position. Similarly, a second fastener 910 is configured to couple the intermediate component 906 with the second component 904 and retain the intermediate component 906 in said position at least partially within the space between the protrusions of the second component 904. The second fastener 910 is configured to be received and extend therethrough the hole on the first protrusion of the second component 904, through a bore of the intermediate component 906, and through the hole of the second protrusion of the second component 904, thus retaining the intermediate component 906 in the aforementioned position (similar to the first component 902). As shown, the first and second components 902, 904 are approximately positioned 90 degrees offset from one another (e.g., out of phase, etc.) when in the engaged orientation with the intermediate component 906. Accordingly, the first and second components 902, 904 are pivotable about the respective first and second fasteners 908, 910 so as to facilitate movement, manipulation, and various positioning of the joint 900.

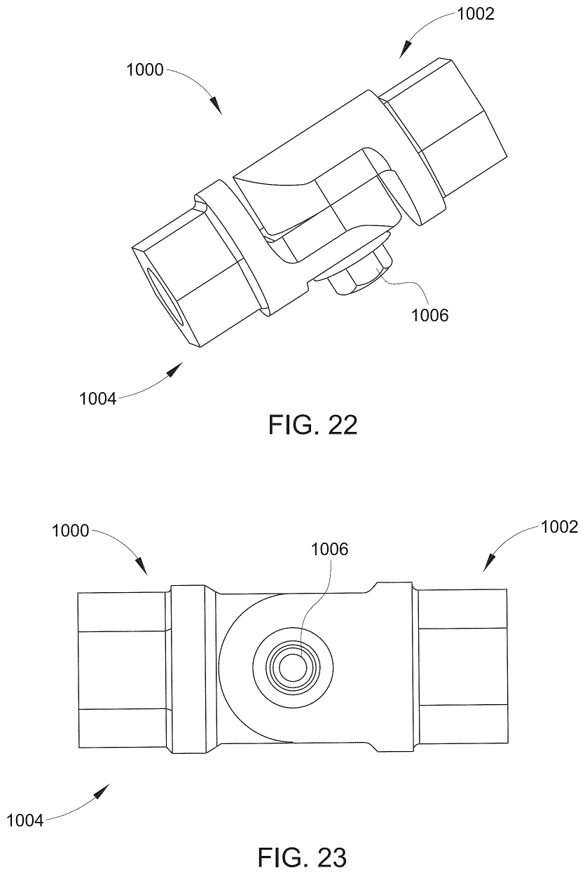

Referring now to FIGS. 22-23, a hinge joint 1000 (referred to hereinafter as “joint 1000”) is shown. Similar to the joints 700, 800, 900, the joint 1000 may be releasably (e.g., threadably, etc.) coupled with one or both ends of the strut 500 (or similar) or other rigid linear components of the system 100 or other similar systems. The joint 1000 may be configured to facilitate the implementation of the system 100 or similar with the strut 500 or other components in “off-axis” configurations including, for example, the “off-axis” configuration described previously with reference to the strut 500 and the joint 700. The joint 1000 is shown to include a first component 1002 and a second component 1004 configured opposite from one another. In some aspects, the first and second components 1002, 1004 may be substantially similar and/or the same component. The first and second components 1002, 1004 are each shown to include a protrusion extending outwardly (parallel to a longitudinal axis thereof) from an end portion and having a bore disposed in a central portion of the protrusion. A fastener 1006 is shown to be received through each of the bores (when the first and second components 1002, 1004 are positioned such that the protrusions abut one another) so as to facilitate a pivoting motion of one or both of the first and second components 1002, 1004 about the fastener 1006.

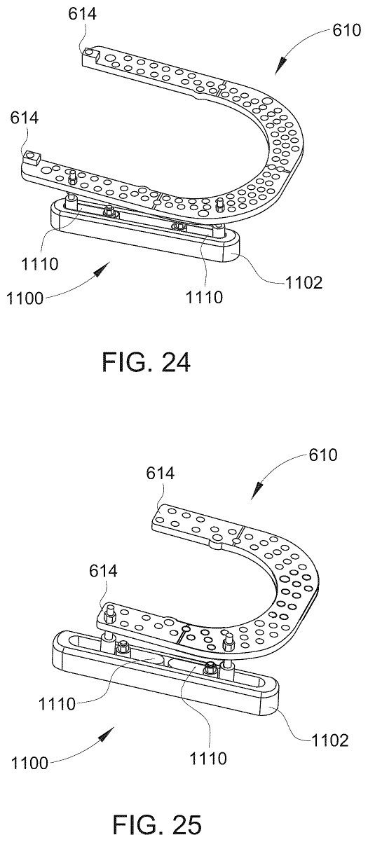

Referring now to FIGS. 24-25, a footplate system 1100 is shown in an exemplary configuration with the third ring member 610 as originally shown as a component of system 100. It should be understood that the footplate system 1100 may be implemented with the third ring member 610 (as well as other components) as an element of the system 100. The footplate system 1100 includes a pair of coupling mechanisms 1110 configured to releasably couple a footplate 1102 with the third ring member 610 (e.g., at least partially adjacent to the terminal ends 614 of the third ring member). As shown, the coupling mechanisms 1110 are translatable within and along a length of a cavity disposed in the footplate 1102 so as to facilitate coupling of any point along the length of the cavity of the footplate 1102 with any portion of the third ring member 610. For example, as shown in FIG. 24, the coupling mechanisms 1110 are positioned at opposite ends of the cavity of the footplate 1102. However, as shown in FIG. 25, the coupling mechanisms 1110 are positioned adjacent one another in a central portion of the cavity of the footplate 1102. Accordingly, the coupling mechanisms 1102 may be positioned and coupled with the third ring member 610 in order to optimize various factors that impact patient comfort and safety including weight distribution, stability, location of the footplate relative to a patient's normal gait, etc.

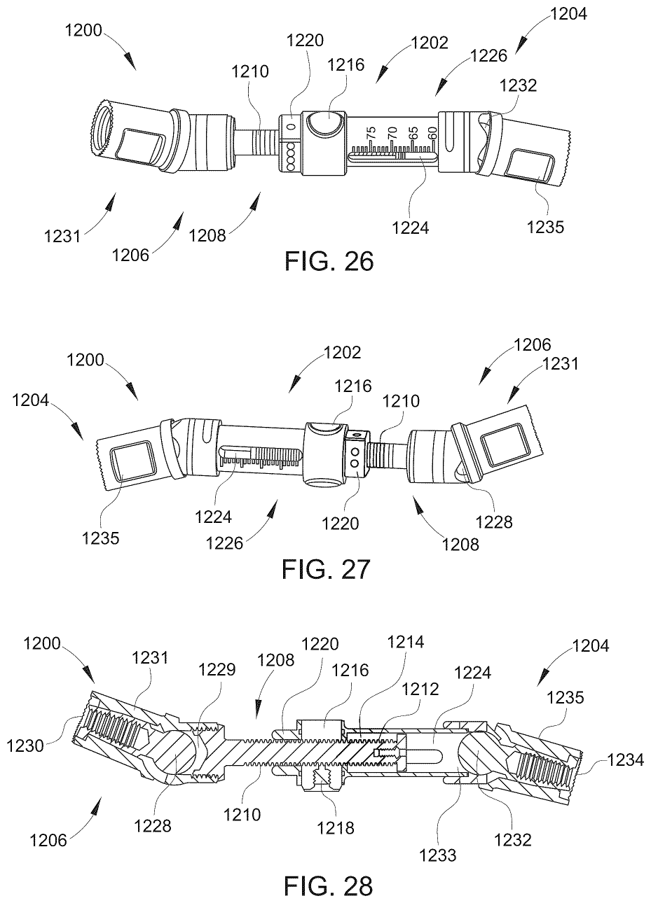

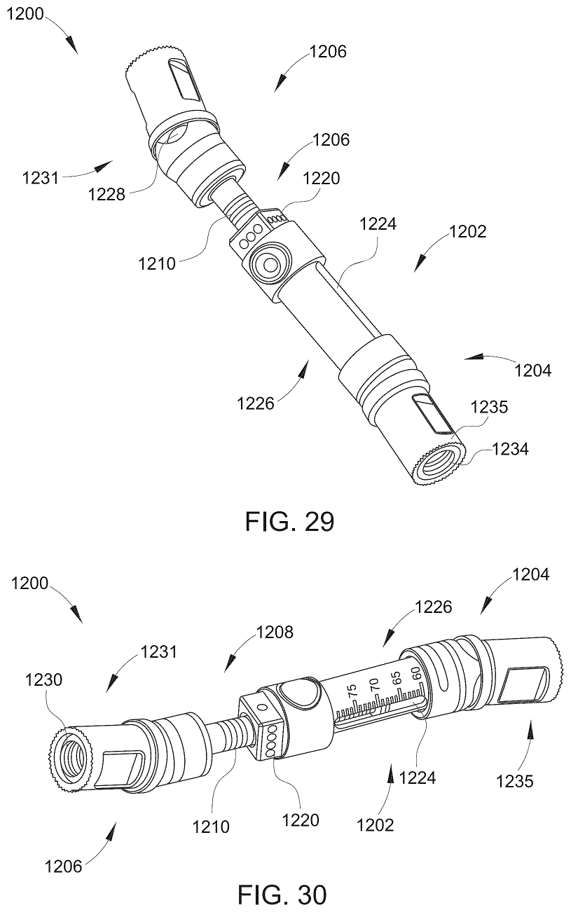

Referring now to FIGS. 26-30, a strut member 1200 (“strut 1200”) is shown, according to an exemplary embodiment. In some aspects, the strut 1200 may be implemented in conjunction with one or more components of an external fixation system, including but not limited to, those shown and described previously herein, for example the system 100 and one or more components therein. For example, the strut 1200 may be releasably couplable with one or more of the ring members 210, 230 in conjunction with one or more bolts, nuts, or other hardware shown and described previously and/or that commonly used in conjunction with orthopedic external fixation systems. Further, one or more struts 1200 may be implemented in conjunction with a system such as the system 100 and may be implemented with one or more other stability, coupling, or fixation members including but limited to static rods or other strut members, for example the strut member 500.

The strut 1200 is shown to include a body 1202 positioned between a first end 1204 and a second end 1206, according to an exemplary embodiment. The body 1202 is shown to include a substantially cylindrical geometry, and as shown in FIG. 28, defines an inner volume 1212 which is shown to be substantially cylindrical. The strut 1200 is further shown to include a translating member 1208, along at least a portion of which a threading 1210 is disposed. The translating member 1208 is shown to have a substantially cylindrical geometry, with each end of the translating member 1208 having a greater lateral geometry (e.g. diameter) than a central portion of the translating member 1208. As shown in FIG. 28, a first end of the translating member 1208 is releasably couplable with the second end 1206 of the strut 1200, while a second end of the translating member 1208 is shown to be positioned within the inner volume 1212 defined by the body 1202. The translating member 1208 is translatable along a longitudinal axis thereof (which may be coaxial with a longitudinal axis of the body 102) such that the second end of the translating member 1208 (that which is disposed within the inner volume 1212) may be translated between a first end 1214 and a second end 1224 of the inner volume 1212. The translating member 1208 is retained within the inner volume 1212 and mechanically limited to a translatable range of motion along the longitudinal axis thereof by the lateral dimension of the second end of the translating member 1208, which is greater than that of any opening to the inner volume 1212.

The body is further shown to include a first actuator 1216, which may include a button as shown in at least FIGS. 26-28. The first actuator 1216 extends circumferentially around the body 1202, with the translating member 1208 extending through a central opening of the first actuator 1216, which is coaxial with the longitudinal axis of the translating member 1208. The first actuator 1216 includes at least one engagement feature 1218, shown in FIG. 28 as a set screw that extends from an outer surface of the first actuator 1216 through to the central opening thereof such that the engagement feature 1218 may be advanced toward the translating member 1218 until the engagement feature 1218 contacts the translating member 1218, thereby retaining it in a desired position relative to the body 1202 and the inner volume 1212. In some aspects, the inner surface first actuator 1216 may include teeth or threads configured to engage with the threading 1210 of the translatable member 1208 so as to retain the translating member 1208 in a desired position along the longitudinal axis of the body 1202.

The body 1202 is shown to include a second actuator 1220, positioned on an end of the body nearest the second end 1206 of the strut 1200. The second actuator 1220 is positioned adjacent the first actuator 1216. As described previously with reference to the first actuator 1216, the first actuator 1216 may be a gross actuation mechanism (e.g., to facilitate larger adjustments of the strut 1200) whereas the second actuator 1220 may be a fine actuation mechanism (e.g., to facilitate smaller adjustments of the strut 1200). The second actuator 1220 is shown to include markers on outer surfaces thereof (e.g., shown to be one, two, three, and four dots on separate faces of the second actuator 1220) indicative of rotational adjustments to increase or decrease the length of the strut 1200 by incrementally translating the translating member 1208 relative to the body 1202. The second actuator 1220 may be configured such that a specific amount of rotation thereof corresponds to specific increase or decrease in the length of the strut 1200 (by translating the translating member 1208 a specific distance). For example, a quarter turn (e.g., 90-degrees about the body 1202) of the second actuator 1220, which may include haptic feedback felt by the user to confirm the quarter turn, may correspond to a 1 mm increase or decrease in the length of the strut by translating the translating member 1 mm in either direction.

The first end 1204 of the strut 1200 is shown to include a first ball joint, the ball 1232 of which is positioned at least partially within an opening 1233 of the body 1202. In some aspects, the opening 1233 may be in fluid communication with the inner volume 1212. The ball 1232 of the first ball joint is shown to include a shaft extending away from the ball 1232, which is disposed at least partially within a housing 1235. The shaft extending away from the ball 1232 is shown to include a cannulation with internal threading 1234 disposed on an inner portion thereof and configured to facilitate coupling with one or more ring members via nuts, bolts, or other hardware commonly implemented in coupling components of orthopedic external fixation systems with each other.

The second end 1206 of the strut 1200 is shown to include a second ball joint, the ball 1228 of which is positioned at least partially within an opening 1229 defined at least partially by a housing 1231 and the first end of the translating member 1208 which, as show in FIG. 28, has a partially concave geometry as a distal-most portion thereof. The ball 1228 of the first ball joint is shown to include a shaft extending away from the ball 1228, which is disposed at least partially within the housing 1231. The shaft extending away from the ball 1228 is shown to include a cannulation with an internal threading 1230 disposed on an inner portion thereof and configured to facilitate coupling with one or more ring members via nuts, bolts, or other hardware commonly implemented in coupling components of orthopedic external fixation systems with each other.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. An external fixation system, comprising:
an upper fixation element comprising a semicircular geometry;
an intermediate fixation element comprising a circular geometry, wherein the intermediate fixation element is releasably coupled with the upper fixation element;
a lower fixation element comprising a substantially U-shaped geometry terminating at a first end point and a second end point, wherein the lower fixation element is releasably coupled with the intermediate fixation element;
an anterior fixation element comprising a semicircular geometry, wherein the anterior fixation element is fixedly coupled with the first and second end points of the lower fixation element; and
at least one strut member releasably coupled with the intermediate fixation element and the lower fixation element.

2. The external fixation system of claim 1, wherein the at least one strut member comprises a pair of strut members.

3. The external fixation system of claim 1, wherein the at least one strut member comprises:
a first end comprising a first threaded portion to facilitate threadable coupling;
a second end comprising a second threaded portion to facilitate threadable coupling; and
a central portion positioned between the first end and the second end.

4. The external fixation system of claim 3, wherein the at least one strut member further comprises:
a gross adjustment mechanism positioned adjacent the second end and the central portion of the at least one strut member, wherein the gross adjustment mechanism comprises:
a collar positioned diametrically about a threaded rod; and
a sleeve positioned diametrically outward from the collar and the threaded rod.

5. The external fixation system of claim 4, wherein the collar is threadably engaged with a threading of the threaded rod such that manipulation of the collar about the threading translates the collar along the rod.

6. The external fixation system of claim 5, wherein the sleeve is translatable along the threaded rod.

7. The external fixation system of claim 6, wherein the system further comprises:
a locking nut translatable along the length of the threaded rod and positioned adjacent the sleeve.

8. The external fixation system of claim 7, further comprising:
a tube, wherein the threaded rod is translatable such that at least a portion of the threaded rod is disposed within the tube.

9. The external fixation system of claim 1, wherein the tube comprises a plurality of markings on an external surface thereof indicating a length of the at least one strut member based on the position of at least a portion of the threaded rod disposed at least partially within the tube.

10. The external fixation system of claim 4, wherein the at least one strut member further comprises:
a fine adjustment mechanism actuatable by rotating at least a portion of the fine adjustment mechanism, wherein rotational manipulation corresponds to predetermined length adjustments to the strut member.

11. A joint of an external fixation system, comprising:
a first component comprising a first longitudinal axis; and
a second component comprising a second longitudinal axis;
wherein the first and second components are manipulatable from a first position in which the first and second longitudinal axes are concentric, to a second position in which the first and second longitudinal axes intersect to form an oblique angle with one another.

12. The external fixation system of claim 11, wherein the first component comprises:
an elongate member comprising first threading on an external surface thereof extending from a terminal end to a central portion thereof; and
a body disposed opposite the elongate member from the terminal end.

13. The external fixation system of claim 12, wherein the body comprises a substantially spherical geometry with a lateral dimension greater than a diameter of the elongate member.

14. The external fixation system of claim 13, wherein the second component comprises a volume extending therethrough, the volume comprising a first threading disposed on at least a portion of an interior surface thereof.

15. The external fixation system of claim 14, further comprising:
an adapter comprising a second threading disposed on an external surface thereof and is configured to engage the first threading of the volume of the second component.

16. The external fixation system of claim 15, wherein the adapter comprises a cannulation and third threading disposed on an interior surface of the cannulation, wherein the third threading is configured to threadably engage a complimentary thread of an element of an external fixation system.

17. The external fixation system of claim 13, wherein the volume of the second component is configured to receive at least a portion of the body therein.

18. The external fixation system of claim 17, wherein the second component comprises a shoulder adjacent the volume configured to retain at least a portion of the body within the volume.

19. The external fixation system of claim 11, wherein the first component is releasably couplable with a ring member of an external fixation system, and wherein the second component is releasably couplable with a strut member of an external fixation system.

20. A joint of an external fixation system, comprising:
a first component comprising:
a first pair of protrusions defining a first space therebetween, wherein each of the first pair of protrusions comprises a first bore extending therethrough, wherein the first bores are coaxial;
a second component comprising:
a second pair of protrusions defining a second space therebetween, wherein each of the second pair of protrusions comprises a second bore extending therethrough, wherein the second bores are coaxial; and
an intermediate component positioned at least partially within the first and second spaces, comprising:
a third bore aligned with the first bores and receiving a first fastener therethrough so as to couple the intermediate component with the first component; and
a fourth bore aligned with the second bores and receiving a second fastener therethrough so as to couple the intermediate component with the second component;
wherein the first and second components are respectively pivotable about the first and second fasteners.
